(19) United States
(12) Patent Application Publication
(10) Pub. No.: US 2016/0199070 A1
(43) Pub. Date: Jul. 14, 2016

(54) PRECISION PICK

(71) Applicant: William Eric MacLeod, Broxton, GA (US)

(72) Inventor: William Eric MacLeod, Broxton, GA (US)

(21) Appl. No.: 14/858,019

(22) Filed: Sep. 18, 2015

Related U.S. Application Data
(63) Continuation of application No. 14/594,641, filed on Jan. 12, 2015.

(51) Int. Cl. A61B 17/16

(52) U.S. Cl. CPC A61B 17/1659

(57) ABSTRACT

A pick device for debriding and preparing a bone surface has a handle, a cannulated debridement shaft and a drill wire. The shaft has an open lumen to receive a drill wire through a proximal end of the handle. The drill wire has a tip end to scrape bone surfaces to create micro-fractures to enhance tissue and cell regeneration. The distal tip can be oriented and provided in a pre-set angle of 30, 45 or 60 degrees relative to the cannulated shaft. The drill wire has a diameter of at least 1.0 mm, preferably in a range of 1 to 3 mm and most preferably 1.45 mm and flexibly bends at the removable distal tip. The drill wire is made of Nitinol, has a stop affixed to the proximal end to limit micro-fracture penetration at the distal end and the distal end has a trocar tip to cut bone tissue.

### RELATED APPLICATIONS

The present invention is a continuation of co-pending U.S. application Ser. No. 14/594,641 filed on Jan. 12, 2015 entitled “Precision Pick”.

### TECHNICAL FIELD

The present invention creates micro-fractures in the exterior surface of bone to act as a scaffold for cells to ensure rapid and consistent cell recruitment when used in cartilage repair procedures.

### BACKGROUND OF THE INVENTION

When repairing cartilage, often there is a defect in the cartilage material and in the adjacent bone. These defects can be in the form of tears or other imperfections that need to be removed and the surrounding tissue prepared prior to any repair of the cartilage.

Recently, there has become a new available cartilage material that lends itself to facilitate rapid cartilage repair and regeneration of the tissue. This material is described in U.S. Pat. No. 8,940,698 issue date Jan. 27, 2015, currently owned by Vivex Biomedical, Inc. This material is provided in strands or branches of fairly large sized particles that when assembled resemble cotton or cotton ball materials which when assembled provide an ideal way for packing a cartilage defect. This material has osteogenic capability and enhances rapid cartilage cell recruitment for the repair of a defect. When packed into a defect, the cartilage material being in the shape of a conformable ball tends to wick or entrap blood and blood clots. This greatly facilitates the ability to repair the cartilage tissue. It has been found useful that when the cartilage defect and the surrounding bone tissue has been properly prepared, the cartilage fluff can generate new cartilage in the defect area very rapidly.

The present invention provides a unique way of facilitating this regeneration by creating micro-fractures in the adjacent bone structure, as described below. These micro-fractures are troughs and valleys scraped into the adjacent bone structure to provide a scaffolding to which cells can easily be attached into these micro-fracture areas.

### SUMMARY OF THE INVENTION

A pick device for debriding and preparing a bone surface. The device has a handle, a cannulated debridement shaft and a drill wire. The cannulated debridement shaft is affixed to the handle. The shaft has an open lumen to receive a drill wire through a proximal end of the handle. The drill wire extends through the handle through the lumen to past a distal tip end of the cannulated shaft. The drill wire has a tip end to scrape bone surfaces to create micro-fractures to enhance tissue and cell regeneration. The distal tip can be oriented and provided in a pre-set angle of 30, 45 or 60 degrees relative to the cannulated shaft. The drill wire has a diameter of at least 1.0 mm, preferably in a range of 1.0 to 3.0 mm and most preferably about 1.45 mm; and flexibly bends at the removable distal tip. The drill wire is made of Nitinol. The drill wire has a stop affixed to the proximal end to limit micro-fracture penetration at the distal end. The distal end has a trocar tip to cut bone tissue.

A method of preparing a repair of a cartilage defect has the steps of removing the defect tissue to create a pocket; scraping the adjacent bone tissue using the pick device; and packing the bone surface and the pocket with cartilage fluff. The method further includes infusing the cartilage fluff with blood.

### BRIEF DESCRIPTION OF THE DRAWINGS

The invention will be described by way of example and with reference to the accompanying drawings in which:

FIG. 1 is a perspective view of the pick device of the present invention.

FIG. 2 is a plan view of the pick device of the present invention.

FIG. 3 is a view of the pick device with the drill wire removed.

FIG. 4 is a plan view of the drill wire.

FIG. 5 is a top view of the pick device of the present invention.

FIG. 6 is a section view taken along line D-D of FIG. 5 of the pick device of the present invention.

### DETAILED DESCRIPTION OF THE INVENTION

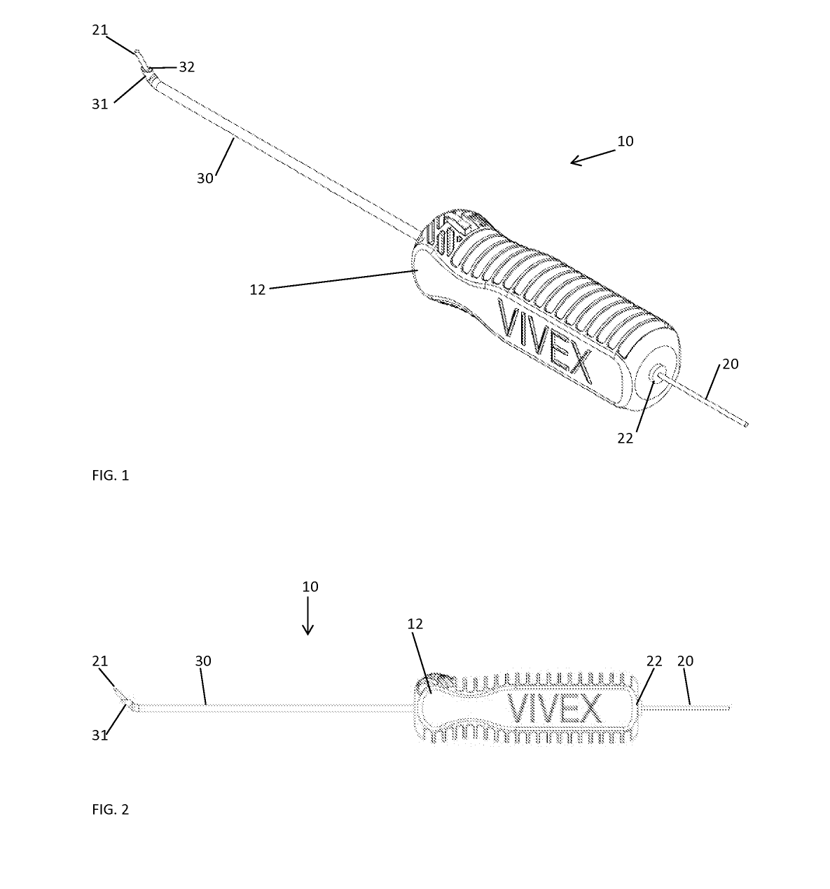

With reference to FIG. 1, the pick device 10 of the present invention is illustrated in a perspective view. The pick device 10 has three major components; a handle 12, a cannulated shaft 30 and a drill wire 20. The handle 12 conveniently allows the surgeon to grip the device 10. The cannulated shaft 30 extends through the handle 12. The cannulated shaft 30 can be provided with a distal end 31. The distal end 31 can be provided straight, or preferably, as shown at an angle. Currently, the pick device 10 is provided with angles of 30, 45 and 60 degrees to facilitate turning the pick device 10 in an orientation that allows forward and aft movement to scrape along the bone structure when a drill wire 20 is inserted through the cannulated lumen 32 of the device 10. As shown, the drill wire 20 has a stop 22 that limits the amount of depth that the drill wire 20 can extend past the distal end of the cannulated shaft 30. As shown, at the distal end a trocar tip 21 is provided. This trocar tip 21 allows scraping of the bone tissue and facilitates creating micro-fractures in that surface. These micro-fractures have been found useful in the recruitment of cells when the cartilage material and adjacent bone has been micro-fractured. In addition, the drill tip 21 allows for drilling micro-fracture holes which would drill small voids into the bone structure.

As illustrated, the distal tip 21 of the drill wire 20 bends slightly, to allow this to happen it has been found useful that the drill wire 20 can be made of a metal material, preferably a Nitinol material so that a pre-disposed angle can easily be achieved by flexing the drill wire 20 as it is pushed through the cannulated opening 32 of the cannulated shaft 30. The cannulated shaft 30 as illustrated has an outside diameter of about 3.175 mm and an inside diameter greater than the diameter of the drill wire 20.

As shown in FIG. 2, a plan view illustrates the distance at which the drill wire 20 extends past the distal end 31 of the cannulated shaft 30. This limits the amount of the depth that the micro-fractures can extend and this is ensured by having a stop 22 at the proximal end of the drill wire 20 abut the handle 12, as illustrated.

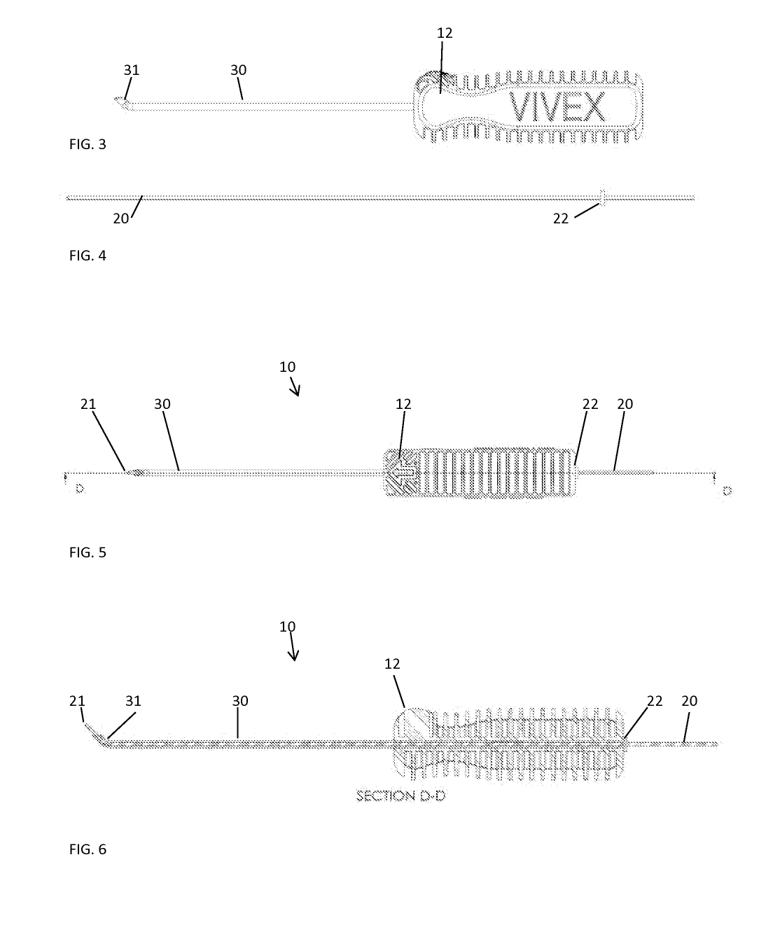

With reference to FIG. 3, the handle 12 and the shaft 30 are shown separate from the drill wire 20 which is shown in FIG. 4. When assembled, as illustrated in FIG. 5 top view, the drill wire tip 21 is shown projecting slightly outward from the tip. FIG. 6 is a section view D-D taken from FIG. 5 shows the handle 12, the shaft 30 and the drill wire 20 when properly positioned.

It has been found that when using this device 10 to repair a cartilage defect, the bone tissue can be scraped creating numerous micro-fractures. These micro-fractures greatly enhance cell recruitment when the defect has been packed with the cartilage material. Historically, cartilage material was provided in small particle form that had no cohesive characteristics. Currently, there is a new product available wherein the cartilage material is provided in branches or strands that are formed in a single ball such that it is ideally suited for placement into a defect. The cartilage material can first be infused with blood of the patient and then positioned into the defect and can be placed into the site to be packed or alternatively, can simply be packed into the defect and the blood of the patient will infuse the material after placement. These features are greatly benefited by the fact that the drill wire has a diameter of at least 1.0 mm, preferably in a range of 1.0 to 3.0 mm and most preferably about 1.45 mm which is larger than would normally be needed to create a micro-fracture. This is done because the strands and branches of the fluff have a large size and ideally benefit from larger troughs or valleys created in the bone structure. When this occurs using the pick device 10 of the present invention, the cartilage fluff has an easy way of nesting onto this surface to provide recruitment of cells for cartilage repair.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

1. A pick device for debriding and preparing a bone surface, the device comprising:
a handle;
a cannulated debridement shaft affixed to the handle and extending to a distal tip end, the distal tip end being angled relative to the cannulated shaft at a pre-set angle, the shaft having an open lumen to receive a drill wire through a proximal end of the handle;
a drill wire extending through the handle through the lumen to past the distal tip end of the cannulated shaft; and
wherein the drill wire has a tip end to scrape bone surfaces to create micro-fractures to enhance tissue and cell regeneration, the micro-fractures being troughs and valleys scraped into the bone surface to form a scaffolding to which the cells can attach, the tip end of the drill wire when the drill wire is assembled through the handle projects slightly outward from the distal tip end sufficient to scrape bone surfaces.

2. The pick device of claim 1 wherein the distal tip end is oriented and provided in a pre-set angle of 30, 45 or 60 degrees relative to the cannulated shaft.

3. The pick device of claim 1 wherein the drill wire has a diameter of at least 1.0 mm, preferably in a range of 1.0 to 3.0 mm and most preferably about 1.45 mm and flexibly bends at the removable distal tip end.

4. The pick device of claim 1 wherein the drill wire is made of Nitinol.

5. The pick device of claim 1 wherein the drill wire has a stop affixed to the proximal end to limit micro-fracture penetration at the distal tip end.

6. The pick device of claim 1 wherein the distal end has a trocar tip to cut bone tissue.

7. A method of preparing a repair of a cartilage defect comprises:
removing a defect tissue to create a pocket exposing adjacent bone tissue;
scraping the adjacent bone tissue using a pick device, the pick device having a handle;
a cannulated debridement shaft affixed to the handle and extending to a distal tip end, the distal tip end being angled relative to the cannulated shaft at a pre-set angle, the shaft having an open lumen to receive a drill wire through a proximal end of the handle;
a drill wire extending through the handle through the lumen to past the distal tip end of the cannulated shaft; and
wherein the drill wire has a tip end to scrape bone surfaces to create micro-fractures to enhance tissue and cell regeneration, the micro-fractures being troughs and valleys scraped into the bone surface to form a scaffolding to which the cells can attach, the tip end of the drill wire when the drill wire is assembled through the handle projects slightly outward from the distal tip end sufficient to scrape bone surfaces; and
packing the bone surface and the pocket with cartilage fluff.

8. The method of claim 7 wherein the method further includes infusing the cartilage fluff with blood.

9. The pick device of claim 1 wherein the drill wire tip end can optionally allow for drilling micro-fracture holes by drilling small voids into the bone structure in addition to scraping.

10. The pick device of claim 2 wherein the distal end of the cannulated shaft has a trocar tip to further assist scraping the bone surface and creating micro-fractures in the bone surface in the form of valleys and troughs for cell attachment.

11. The pick device of claim 3 wherein the drill wire has a diameter in the range of 1.0 to 3 mm.

12. The pick device of claim 11 wherein the drill wire diameter is 1.45 mm.
